(19) United States
(12) Patent Application Publication
(10) Pub. No.: US 2009/0215733 A1
(43) Pub. Date: Aug. 27, 2009

(54) THERAPY FOR THE PROPHYLAXIS OR TREATMENT OF ADVERSE BODY COMPOSITION CHANGES AND/OR DECREASED MUSCLE STRENGTH AFTER ANDROGEN OR GNRH ANALOGUE INTAKE

(76) Inventor: Michael Charles Scally, Houston, TX (US)

Correspondence Address: ERIC P. MIRABEL, 35 TECHNOLOGY DRIVE, SUITE 100, WARREN, NJ, 07059, US

(21) Appl. No.: 12/391,338

(22) Filed: Feb. 24, 2009

Related U.S. Application Data
(60) Provisional application No. 61/031,842, filed on Feb. 27, 2008.
(60) Provisional application No. 61/031,461, filed on Feb. 26, 2008.

(51) Int. Cl. A61K 31/566; A61K 31/138; A61K 31/4196

(52) U.S. Cl. 514/171; 514/651; 514/383

(57) ABSTRACT

Methods of treating or reducing the risk of one or more effects or signs of induced hypogonadism after androgen or GnRH intake are disclosed herein. In an embodiment, a method of treating one or more effects after androgen or gonadotropin releasing hormone (GnRH) analogue intake comprises administering to a male subject at least one of a blocking compound which antagonizes estradiol binding to estradiol receptors or an inhibitor compound which inhibits endogenous production of estradiol, after androgen or gonadotropin releasing hormone (GnRH) analogue intake, to treat the one or more effects. The one or more effects include adverse body composition changes, decreased muscle strength, or both. The inhibitor compound and/or blocking compound may include antiestrogen agents, aromatase inhibitors, or combinations thereof.

### CROSS-REFERENCE TO RELATED APPLICATIONS

This application claims the benefit of Provisional Application No. 61/031,842 filed on Feb. 27, 2008 and Provisional Application No. 61/031,461 filed Feb. 26, 2008.

### BACKGROUND

1. Field of the Invention

This invention relates to treating adverse body composition changes and decreased muscle strength from induced hypogonadism after androgen or gonadotropin releasing hormone (GnRH) analogue intake.

2. Background of the Invention

The hypothalamic pituitary testicular axis (HPTA) is the homeostatic system responsible for maintaining, supporting, and ensuring reproduction, bone density, muscle mass, and other important and vital physiological and psychological processes, and is maintained through active in vivo monitoring of hormone levels and feedback.

Structural components of the HPTA are the hypothalamo-pituitary, testicles, and androgen receptor (AR) located on certain end organs (prostate, bone, and muscle). The major hormones monitored by the hypothalamic pituitary testicular axis are gonadotropin releasing hormone (GnRH), luteinizing hormone (LH), follicle-stimulating hormone (FSH), inhibin, testosterone, dihydrotestosterone (DHT), and estradiol. There must be a level of communication between the hypothalamo-pituitary, testes, and androgen receptor (AR) to maintain HPTA homeostasis. The HPTA has two components, both spermatogenesis and testosterone production. They are not equivalent and, in fact, have two very separate hormonal processes for homeostasis. Absent FSH, there is no testicular spermatozoa production. Absent LH, there is no testicular testosterone production.

In males, the pulsatile secretion of gonadotropin releasing hormone (GnRH) from the hypothalamus stimulates LH and FSH secretion. Luteinizing hormone (LH) secretion by the pituitary positively stimulates testicular testosterone (T) production. Follicle-stimulating hormone (FSH) in the presence of testosterone stimulates testicular spermatogenesis. The secretion of GnRH, FSH, and LH is regulated by negative feedback from endogenous testosterone and estradiol as monitored by the hypothalamo-pituitary.

The regulation of the feedback of luteinizing hormone (LH) and follicle-stimulating hormone (FSH) in men is, in part, under the control of estradiol, which results from the aromatization of testosterone. The biosynthesis of estrogens from C19 steroids is by the enzyme aromatase cytochrome P450. This enzyme converts androstenedione and testosterone to estrone and estradiol, respectively.

Estrogens contribute substantially to the negative feedback regulation of gonadotropin secretion. A great part, if not all, of the inhibitory effect on gonadotropin secretion is mediated by the endogenous conversion of testosterone to estradiol. The restraining action of estrogens on gonadotropin secretion in men is exerted both at the pituitary and at the hypothalamic levels. Estradiol has a much larger, inhibitory effect than testosterone, being 200-fold more effective in suppressing LH secretion.

Hypogonadism is a disturbance of HPTA homeostasis caused by inadequate gonadal function, and manifested by deficiencies in spermatogenesis or the secretion of testosterone. Hypogonadism is defined by either reduced reproductive capacity, infertility, or biochemically, by reduced testosterone levels.

Hypogonadism has potentially serious consequences that might include but are not limited to adverse body composition changes (decrease muscle mass and increased adiposity), decreased muscle strength, infertility, bone loss (osteoporosis), increase in cardiovascular risk, mood disturbances (depression, anger, low self esteem, sense of well-being, guilt, increased stress, and anhedonia), sexual dysfunction (decreased libido, decreased spontaneous erections, decreased ejaculate, erection dysfunction, decreased sexual fantasies, and anorgasmia), decreased cognitive testing (memory and concentration), sleep disturbances, and constitutional symptoms (general fatigue, agitation, motor dyskinesia, and decreased appetite).

Use of GnRH analogues result in induced hypogonadism by their effects on gonadotropin levels. A GnRH agonist acts as a potent inhibitor of gonadotropin secretion when given continuously in therapeutic doses. Treatment with gonadotropin-releasing hormone (GnRH) analogues inhibits pituitary secretion of LH and thus testicular production of testosterone. Chronic administration of GnRH agonist results in suppression of testicular steroidogenesis, androgen deprivation therapy (ADT). ADT is a form of therapy in the treatment of prostate cancer that is androgen-sensitive. Prostate cancer is one of the most frequently occurring cancers among men in the United States, with hundreds of thousands of new cases diagnosed each year.

GnRH induced hypogonadism is associated with adverse body composition changes. Induced hypogonadism studies show that a result of GnRH analogues is a loss of muscle mass (decreased lean body mass), increased adiposity, and decreased muscle strength.

Androgen administration results in a form of induced hypogonadism called functional hypogonadotropic hypogonadism. Androgen-induced hypogonadism (AIH) is the functional incompetence of the testes with subnormal or impaired production of testosterone or spermatozoa due to administration of androgens or anabolic steroids. Nonsteroidal androgen administration is currently in the research and investigational stages. Studies on nonsteroidal androgens indicate that their clinical use will result in androgen-induced hypogonadism by their effects on gonadotropin levels. Androgen administration blocks the production of hypothalamic GnRH, this, in turns, shuts down the LH and FSH secretion by the pituitary. This, in turn, reduces the testosterone production by Leydig cells in the interstitium, which along with reducing intratesticular androgen levels and decreased FSH secretion leads to aspermatogenesis. The neuroendocrine abnormality in AIH must be distinguished from classical forms of GnRH deficiency, idiopathic hypogonadotropic hypogonadism (IHH), where the defect in GnRH secretion is clearly permanent rather than transient. Histology of the testicular tissue indicate a reduction in seminiferous tubular diameter, the arrest of advanced spermatogenesis, decrease in the number of spermatocytes and spermatids, Sertoli cell apoptosis, and a depletion of the number of Leydig cells in the interstitial compartment. These are accompanied by changes in semen parameters and testis atrophy.

Earlier studies demonstrate the improvements in body composition obtained during androgen administration, are lost after androgen cessation. In 1990, the World Health Organization study tested the use of testosterone enanthate 200-mg intramuscular/week for 12 months as a male contraceptive. In the WHO male contraception study, after starting testosterone injections, there were increases in body weight that returned to baseline in the recovery period. In 1992, Forbes et al. published a study describing the sequence of changes in body composition induced by testosterone and reversal of changes after drug cessation. Upon testosterone discontinuation, LBM progressively declined at a rate that suggests that half of the maximum increment was lost in about 2 months. In 2004, a randomized controlled study reported on the body composition changes during administration and after a twelve-week follow-up period after AAS cessation. The study found that the positive body composition changes in lean body mass, muscle area, and strength produced by the androgen in the study had completely disappeared twelve weeks after AAS cessation.

Government health agencies (as evidenced by the labels and packaging inserts associated with FDA approved androgens) and the medical and research community (as evidenced by a lack of exploratory studies, papers and research grants) do not recognize the period after prescribed androgens to be associated with any clinically significant adverse events.

A particular androgen, oxandrolone, is currently FDA approved as adjunctive therapy to promote weight gain after weight loss following extensive surgery, chronic infections, or severe trauma, and in some patients who without definite pathophysiologic reasons fail to gain or to maintain normal weight, to offset the protein catabolism associated with prolonged administration of corticosteroids, and for the relief of the bone pain frequently accompanying osteoporosis. Androgens are also FDA approved and prescribed for anemia.

In addition to those FDA approved indications for oxandrolone, clinical studies utilizing androgens, inclusive of testosterone, have received particular attention with regard to improving body composition and muscle strength in those with chronic illness. Androgen use for these purposes is of a limited duration.

Clinical studies utilizing androgen administration include but are not limited to the elderly (sarcopenia is the loss of muscle mass and muscle strength in ageing), end stage renal disease (ESRD) on hemodialysis, chronic obstructive lung disease (COPD), HIV+ males, alcoholic hepatitis, glucocorticoid induced osteoporosis, and others.

According to the U.S. reports the Centers for Disease Control and Prevention, there are over 500,000 HIV+ males in the United States. A significant percentage of these individuals receive androgen treatment to effect positive body composition changes (increase muscle mass and decreased adiposity) and increase muscle strength. Those experiencing lean muscle wasting greater than 10% will likely be administered a form of androgen therapy to help retain fat free tissue.

Another disease that has been treated with androgen is chronic obstructive pulmonary disease (COPD). COPD refers to two lung diseases, those being chronic bronchitis and emphysema. Both of these conditions frequently co-exist, hence physicians prefer the term COPD. COPD is the fourth leading cause of death in America. In 2004, estimates are that 11.4 million U.S. adults (aged 18 and over) have COPD.

Androgen treatment has also found use in the treatment of osteoporosis. As of 2002, over 14 million men suffer with osteoporosis and low bone mass according to the National Osteoporosis Foundation.

These studies alone results in a very large population receiving androgen treatment. The translation of the clinical studies to direct patient care easily results in androgen administration to millions of individuals.

The disorders above are associated with a considerable degree of morbidity and mortality. Any additional comorbid disease will undoubtedly lead to additional adverse outcomes, not less. In already compromised individuals, hypogonadism, whether or not induced, is a disease with associated particularly significant adverse events that is clearly such a comorbid condition. Published studies utilizing prescribed androgen therapy do not include the follow-up period after androgen intake. This period might potentially leave them in a state of health worse than when first prescribed androgen.

Antiestrogens interfere with the normal negative feedback of estradiol at hypothalamic and pituitary levels in order to increase endogenous gonadotropin-releasing hormone secretion from the hypothalamus and LH secretion directly from the pituitary. In turn, LH stimulates Leydig cells in the testes, and this leads to increased local testosterone production.

Studies demonstrating a significant increase of gonadotropin levels under aromatase inhibition illustrate the important contribution of estrogens to the sex steroid feedback inhibition of gonadotropin secretion in men. Pharmacological inhibition of aromatase activity results in increased levels of gonadotropin and testosterone levels.

Declining, or suppressed, circulating testosterone levels because of induced hypogonadal conditions has many negative consequences in males. There is a direct association between induced hypogonadism (decreased levels of testosterone) and a number of effects, notably adverse body composition changes and decreased muscle strength. Consequently, there is a need for alternative methods of treating adverse body composition changes and decreased muscle strength after androgen or GnRH analogue intake.

New innovative approaches are urgently needed at both the basic science and clinical levels to treat effects such as adverse body composition changes (loss of muscle mass and increased adiposity) and decreased muscle strength in a male subject with induced hypogonadism due to either androgens or GnRH analogue intake. To date, no methods have been proposed to treat such effects of induced hypogonadism after androgen or GnRH analogue intake. Accordingly, methods of treating or reducing the risk of one or more effects or signs of induced hypogonadism after androgen or GnRH analogue intake are disclosed herein.

### SUMMARY

In an embodiment, a method of treating one or more effects of induced hypogonadism in a male subject after androgen or gonadotropin releasing hormone (GnRH) analogue intake, the method comprises administering to the male subject at least one of an antiestrogen agent or an aromatase inhibitor, after androgen or GnRH analogue intake so as to reduce the risk of acquiring one or more effects due to induced hypogonadism after androgen or GnRH analogue intake. The one or more effects include adverse body composition changes, decreased muscle strength, or both.

In another embodiment, a method of reducing the risk of acquiring one or more effects of induced hypogonadism in a male subject after androgen or GnRH analogue intake, the method comprises administering to the male subject at least one of an antiestrogen agent or an aromatase inhibitor, after androgen or GnRH analogue intake so as to reduce the risk of acquiring one or more effects due to induced hypogonadism after androgen or GnRH analogue intake. The one or more effects include adverse body composition changes, decreased muscle strength, or both.

The foregoing has outlined rather broadly the features and technical advantages of the invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### Notation and Nomenclature

Certain terms are used throughout the following description and claims to refer to particular compounds and components, but these terms should be read as encompassing compounds and components with the same function, even if such compounds and components are sometimes herein or elsewhere named differently.

In the following discussion and in the claims, the terms “including” and “comprising” are used in an open-ended fashion, and thus should always be interpreted to mean “including, but not limited to . . . ”.

### DETAILED DESCRIPTION OF THE PREFERRED EMBODIMENTS

In an embodiment of a method for treating one or more effects of induced hypogonadism after androgen or GnRH analogue intake comprises administering a blocking compound which antagonizes or inhibits estradiol binding to the estradiol receptors and/or an inhibitor compound which inhibits endogenous production of estradiol. Embodiments of the disclosed method may also be used for prevention of one or more effects of induced hypogonadism after androgen or GnRH analogue intake.

In some embodiments, the method includes administering one or more androgens, or administering GnRH analogue to a male subject, and then administering the blocking compound and/or inhibitor compound after androgen or GnRH analogue intake. More specifically, the blocking compound and/or inhibitor compound may include at least one of an antiestrogen agent, an aromatase inhibitor, or combinations thereof. The one or more effects of induced hypogonadism include adverse body composition changes and/or decreased muscle strength. It is emphasized that the disclosed methods will be used to treat the specific effects of induced hypogonadism rather than the hypogonadism itself, which has never been done before.

As used herein, the term “adverse body composition changes” may refer to any unfavorable change in the body composition of a subject or a patient that may be detrimental to the subject's health. Furthermore, as used herein, “body composition” may refer to a subject's proportion of muscle mass and/or proportion of body fat. More specifically, an adverse body composition change or decrease in muscle strength may comprise levels or values consider statistically significant within peer-reviewed literature. In one embodiment, an adverse body composition change may comprise a change in body composition (e.g. loss of muscle mass or increase in body fat) or a change in muscle strength with a significance p level of 0.05 or greater.

As used herein, the term “androgen” may refer to any natural or synthetic compound that controls that stimulates or controls the development and maintenance of masculine characteristics in a mammal. The androgen may be steroidal or nonsteroidal. Steroidal androgens include without limitation testosterone, nandrolone, oxandrolone, oxymetholone, stanazolol, dehydroepiandrosterone, androstenediol, androsterone, dihydrotestosterone, or combinations thereof. GnRH refers to GnRH itself or any of its analogues and/or derivatives. The antiestrogen agent and/or aromatase inhibitor is administered to treat adverse body composition changes or decreased muscle strength after androgen or GnRH analogue intake.

In one embodiment, the disclosed methods may be used for treatment or prevention of adverse body composition changes or decreased muscle strength after prescribed use androgens or GnRH analogue for a medically recognized disease or condition. Heretofore, no methods for treating adverse body composition changes or decreased muscle strength have been available for patients after medically prescribed androgens or GnRH analogue. Accordingly, embodiments of the method may be used after prescribed treatment with androgens and/or GnRH analogue for a condition or disease such as without limitation, human immunodeficiency virus (HIV), prostate cancer, osteoporosis, obesity, sarcopenia, end stage renal disease (ESRD), chronic obstructive lung disease (COPD), alcoholic hepatitis, or combinations thereof.

Without being limited by theory, it is believed that the administration of blocking compounds and/or inhibitor compounds (e.g. antiestrogens or aromatase inhibitors) interferes with the normal negative feedback of estradiol at hypothalamic and pituitary levels in order to increase endogenous gonadotropin-releasing hormone secretion from the hypothalamus and LH secretion directly from the pituitary. In turn, LH stimulates Leydig cells in the testes, and this leads to increased local testosterone production. In other words, again without being bound by theory, these compounds antagonize or inhibit estradiol binding to the estradiol receptors to inhibit the action of estradiol.

As described above, at least one of the blocking compounds and/or inhibitor compounds is administered to treat adverse body composition changes or decreased muscle strength after androgen or GnRH analogue intake. Specifically, adverse body composition changes include without limitation, adverse body composition changes such as muscle loss, increased body fat or adiposity, or combinations thereof.

Any techniques and methodologies known to those of skill in the art may be used to determine muscle loss, increased body fat, muscle wasting, or decreased muscle strength. These include but are not limited to body mass index (BMI), anthropometry, air-displacement plethysmography, bioimpedance analysis (BIA), magnetic resonance imaging (MRI), computerized tomography (CT), dilution techniques (total body water using deuterium isotope analysis or extracellular fluid using sodium bromide dilution), dual energy-ray absorptiometry (DEXA), hydrodensitometry (underwater weighing), and 40 k whole body counting.

In one embodiment, body composition may be measured using the four-compartment (4C) model in which fat free body tissue is divided into its constituent parts, namely water, protein and mineral. The 4C model then incorporates independent measurements of mineral, total body water and body density to derive body fat. The 4C model is often used as a criterion method to compare the accuracy of other methods for assessing body fat.

Body mass index (BMI) may be another is another expression of height and weight to characterize body fatness where BMI=weight (kg) divided by height (squared meters). This value may provide a fast and inexpensive method of evaluation. The main assumption of BMI guidelines is that body mass, adjusted for stature squared, is closely associated with body fatness and consequent morbidity and mortality. The BMI classification has been utilized by the WHO report and the NIH evidence report because of its ease in calculation and availability of variables (height and weight).

In another embodiment, bioelectrical impedance analysis (BIA) may be used for estimating body composition. BIA actually determines the electrical impedance, or opposition to the flow of an electric current, of body tissues, which can be used to calculate an estimate of total body water (TBW). TBW can be used to estimate fat-free body mass and, by difference with body weight, body fat.

Dual energy x-ray absorptiometry (DEXA) is another method that may be used for body composition determination. The instrument uses X-rays of 2 distinct energy levels that are attenuated to different extents by fat, bone, and lean mass. Subsequently, the differential attenuation of the two energies is utilized to quantify bone, lean, and/or fat tissue. DEXA provides for an accurate assessment of soft tissue composition or its change.

A number of methodologies or techniques may be used to evaluate or determine loss of muscular strength. These include but are not limited to use of an instrument known as the Jackson Isometric Strength Evaluation System (Lafayette Instruments, Inc.), handgrip strength, back and leg strength, one repetition maximum strength test (1 RM), six-minute walk test, standing balance test, and the chair stand.

The blocking compound and/or inhibitor compound may be administered in an amount effective to treat adverse body composition changes or decreased muscle strength after androgen or GnRH analogue intake. The blocking compound and/or inhibitor compound may also be administered in an amount effective to reduce the risk of acquiring adverse body composition changes or decreased muscle strength after androgen or GnRH analogue intake. Dose and duration of medications may not depend on patient weight. Additionally, the treatment dose and duration with antiestrogen or aromatase inhibitor may not be affected by the androgen or GnRH analogue type, dose, and duration. The antiestrogen or aromatization inhibitor may be administered once or twice a day. Specific dosages and duration of treatment to maximize efficacy can be derived in human clinical trials or extrapolated from animal studies (based on human/animal weight ratios or other known relationships of the animal model to human dosages).

The antiestrogen(s) may be administered at a daily dosage ranging from 10 mg to 40 mg, or from 10 mg to 80 mg, or from 25 mg to 200 mg, or from 20 mg to 80 mg. The aromatase inhibitor(s) may be administered at a daily dosage ranging from 1 mg to 5 mg, or 0.5 mg to 2.0 mg, or from 12.5 mg to 50 mg, or from 125 mg to 500 mg. In addition, the blocking compound and/or inhibitor compound may be administered for a time ranging from 1 day to 15 days, alternatively from 1 day to 30 days, alternatively from 1 day to 45 days, alternatively from 1 day to 60 days, alternatively from 1 day to 90 days.

In an embodiment, the antiestrogen agent comprises enclomiphene. Suitable dosages of enclomiphene citrate may range from 12.5 to 100 mg per day.

In another embodiment, the antiestrogen agent comprises the combination of clomiphene and tamoxifen, which is a trans-isomer of a triphenylethylene derivative. Chemically, tamoxifen has the following chemical formula: (Z)2-[4-(1,2-diphenyl-1-butenyl)phenoxy]-N,N-dimethylethanamine 2-hydroxy-1,2,3-propanetricarboxylate (1:1). Suitable dosages of clomiphene and tamoxifen may range from 50 to 100 mg per day and 10 to 40 mg per day, respectively.

In another embodiment, the antiestrogen agent comprises 4-hydroxytamoxifen, the active metabolite of tamoxifen. Suitable dosages of 4-hydroxytamoxifen range from 10 to 40 mg per day.

In yet another embodiment, the antiestrogen agent comprises toremifene, a nonsteroidal inhibitor of estrogen synthesis. More specifically, toremifene is an example of a triphenylalkylene compound described in U.S. Pat. Nos. 4,696,949 and 5,491,173, which are incorporated herein by reference. Suitable dosages of toremifene may range from 20 to 80 mg per day.

In one embodiment, the method comprises administering at least one aromatase inhibitor. Aromatase cytochrome P450 (CYP19) is enzyme responsible for the synthesis of estrone and estradiol from androstenedione and testosterone, respectively. In an embodiment, the aromatase inhibitor agent comprises the nonsteroidal triazole anastrozole (Arimidex®). Anastrozole is a novel, nonsteroidal aromatase inhibitor [1,3-benzenediacetonitrile, α,α,α′,α′-tetramethyl-5-(1H-1,2,4-triazol-1-ylmethyl)] that blocks the conversion of Δ4-androstenedione to estrone and of testosterone to estradiol. Anastrozole may be found under the trade name Arimidex (anastrozole, AstraZeneca, Wilmington, Del.). Suitable dosages of anastrozole range from 0.5 to 2.0 mg per day.

In another embodiment, the aromatase inhibitor agent comprises the nonsteroidal aromatase inhibitor, triazole letrozole (Femarao). It has the following chemical formula: 4,4′-(1H-1,2,4-Triazol-1-ylmethylene)dibenzonitrile. Suitable dosages of letrozole may range from 1.0 to 5.0 mg per day.

In another embodiment, the aromatase inhibitor agent comprises the steroid formestane (4-hydroxy-4-androstene-3,17-dione), a selective aromatase inhibitor. Suitable dosages of formestane may range from 125 to 500 mg per day.

In another embodiment, the aromatase inhibitor agent comprises the oral steroidal aromatase inhibitor, exemestane (trade name Aromasino). Suitable dosages of exemestane may range from 12.5 to 50 mg per day.

It is further contemplated that any analog, derivative, isomer, metabolite, pharmaceutically acceptable salt, pharmaceutical product, hydrate, or N-oxide of the above disclosed antiestrogens and/or aromatase inhibitors may be administered to a patient. Pharmaceutically acceptable salts include without limitation, acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Suitable salts may be formed from the free carboxyl groups and be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Administering the disclosed compounds may comprise any methods known to those of skill in the art. More specifically, the disclosed compounds may be administered orally in solid form (i.e. pill) or liquid form (i.e. syrup), or alternatively, may be administered topically through the application to the skin in the form of a cream or lotion. Suitable solid oral formulations include without limitation, tablets, capsules, pills, granules, pellets, etc. Suitable liquid oral formulations include without limitation, solutions, suspensions, dispersions, emulsions, oils, etc. The disclosed compounds (i.e. antiestrogen, aromatase inhibitor) may be part or an ingredient of a pharmaceutical composition such as without limitation, a pellet, a tablet, a capsule, a solution, a suspension, an emulsion, an elixir, a gel, a cream, a suppository or a parenteral formulation. Furthermore, the antiestrogen and/or aromatase inhibitor may be administered intravenously, intraatrially, or intramuscularly in liquid form via a syringe, intravenous line, or other known means.

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims which follow, that scope including all equivalents of the subject matter of the claims.

Experimental Details Section

### EXAMPLE 1

### Using Enclomiphene to Positively Influence Lean Body Mass, Fat Mass, and Muscle Strength in HIV+ Human Male Subjects

Enclomiphene's efficacy in treating the adverse body composition changes and decreased muscle strength due to androgen induced hypogonadism will be verified in HIV+ male subjects currently prescribed androgens for their positive anabolic effects. They will be administered Enclomiphene upon androgen cessation at a dosage of 30 mg, twice per day, for 90 days. Evaluable data is baseline and 90-day lean body mass, fat mass, and muscle strength.

### EXAMPLE 2

### Using Enclomiphene to Positively Influence Lean Body Mass, Fat Mass, and Muscle Strength in Human Male Subjects with Prostate Cancer

Enclomiphene's efficacy in treating the adverse body composition changes and decreased muscle strength due to GnRH induced hypogonadism will be verified in prostate cancer subjects administered GnRH analogues as androgen deprivation therapy. They will be administered Enclomiphene upon cessation of GnRH analogue administration at a dosage of 30 mg, twice per day, for 90 days. Evaluable data is baseline and 90-day lean body mass, fat mass, and muscle strength.

The discussion of a reference is not an admission that it is prior art to the present invention, especially any reference that may have a publication date after the priority date of this application. The disclosures of all patents, patent applications, and publications cited herein are hereby incorporated herein by reference in their entirety, to the extent that they provide exemplary, procedural, or other details supplementary to those set forth herein. The specification and description above are exemplary and not intended to be limiting, and the invention is defined only in the claims which follow and includes all equivalents of the subject matter of the claims.

What is claimed is:

1. A method of treating, preventing or inhibiting adverse body composition changes, decreased muscle strength, or both from induced hypogonadism after androgen or gonadotropin releasing hormone (GnRH) analogue intake, the method comprising:
administering to a subject at least one of a blocking compound which antagonizes or inhibits estradiol binding to estradiol receptors and an inhibitor compound which inhibits endogenous production of estradiol.

2. The method according to claim 1, wherein the adverse body composition changes are selected from the group consisting of loss of muscle mass, increased body fat, and combinations thereof.

3. The method according to claim 1 wherein the blocking compound is an antiestrogen and the inhibitor compound is an aromatase inhibitor.

4. The method according to claim 3, wherein the antiestrogen comprises an analog, a derivative, isomer, metabolite, pharmaceutically acceptable salt, pharmaceutical product, hydrate, N-oxide, or any combination thereof, of the antiestrogen.

5. The method according to claim 3, wherein the aromatase inhibitor comprises an analog, a derivative, an isomer, a metabolite, a pharmaceutically acceptable salt, a pharmaceutical product, a hydrate, an N-oxide, of the aromatase inhibitor.

6. The method according to claim 3, wherein the antiestrogen is selected from the group consisting of clomiphene, enclomiphene, tamoxifen, 4-hydroxytamoxifen, toremifene, and combinations thereof.

7. The method according to claim 3, wherein the aromatase inhibitor is selected from the group consisting of letrozole, anastrozole, formestane (4-hydroxy-4-androstene-3,17-dione), exemestane, and combinations thereof.

8. The method of claim 3, wherein the antiestrogen is clomiphene or enclomiphene administered at a dosage from 10 mg to 200 mg/day.

9. The method of claim 3, wherein the antiestrogen is tamoxifen administered at a dosage from 10 mg to 40 mg/day.

10. The method of claim 3, wherein the antiestrogens are clomiphene and tamoxifen administered at dosages of 50 mg and 10 mg, respectively, twice per day.

11. The method of claim 5, wherein the aromatase inhibitor is anastrozole administered at a dosage of from 0.5 to 2.0 mg per day.

12. The method of claim 5, wherein the aromatase inhibitor is letrozole administered at a dosage of from 1.0 to 5.0 mg per day.

13. The method of claim 5, wherein the aromatase inhibitor is formestane, administered at a dosage of from 125-500 mg per day.

14. The method of claim 5, wherein the aromatase inhibitor is exemestane administered at a dosage of from 12.5-50 mg per day.

15. The method according to claim 1, wherein at least one of the blocking compound or the inhibitor compound is administered to the male subject for a period ranging from about 1 day to about 90 days.

16. The method according to claim 1, wherein the administering to a male subject at least one of a blocking compound which antagonizes estradiol binding to estradiol receptors or an inhibitor compound which inhibits endogenous production of estradiol comprises intravenously, intraarterially, subcutaneously, or intramuscularly injecting at least one of the blocking compound or the inhibitor compound, or orally ingesting them, or topically applying them.

17. The method of claim 8 wherein the antiestrogen is enclomiphene administered upon cessation of after androgen or gonadotropin releasing hormone (GnRH) analogue intake at a dosage of 30 mg, twice per day, for 90 days.
